(19) United States
(12) Patent Application Publication
(10) Pub. No.: US 2025/0009815 A1
(43) Pub. Date: Jan. 9, 2025

(54) COMPOSITION FOR COMPETITIVE INHIBITION OF PATHOGENS AND RESTORATION OF MICROBIAL ECOLOGICAL BALANCE

(71) Applicant: Sanzyme Biologics Private Limited, Hyderabad (IN)

(72) Inventors: Raunak Jay Soman, Hyderabad (IN); Venkat Swamy Malisetty, Hyderabad (IN); Jay Soman, Hyderabad (IN)

(73) Assignee: Sanzyme Biologics Private Limited, Hyderabad (IN)

(21) Appl. No.: 18/894,279

(22) Filed: Sep. 24, 2024

Related U.S. Application Data
(63) Continuation-in-part of application No. 16/788,990, filed on Feb. 12, 2020, now Pat. No. 12,097,225.

(30) Foreign Application Priority Data
Feb. 12, 2019 (IN) 201941005414

(51) Int. Cl. A61K 35/742; A61K 31/192; A61K 31/194; A61P 15/02; A61P 31/04; A61P 31/10

(52) U.S. Cl. CPC A61K 35/742; A61K 31/192; A61K 31/194; A61P 15/02; A61P 31/04; A61P 31/10

(57) ABSTRACT

Disclosed are compositions for competitive inhibition of pathogens against pathogenic infections comprising (a) Bacillus coagulans; (b) Bacillus subtilis; (c) citric acid; and (d) gallic acid including any combinations thereof. The compositions disclosed in this invention is capable to treat and prevent pathogenic infections of the genital organs as well as suitable for treatment and prevention of dermal infections. A method of preventing recurrence of the infections by way of supporting the growth of inherent natural doderlein microbial populations due to application of the compositions of the present invention are also disclosed. Further, use of the compositions of the present invention obviates the need of prior specific diagnosis of the infections due to its broad-spectrum activity against multiple bacterial and fungal pathogens.

This application claims priority to Indian Provisional application 20/194,1005414, filed on Feb. 12, 2019, and is a Continuation-In-Part application to patent application U.S. Ser. No. 16/788,990 filed on Feb. 12, 2020, to be issued as U.S. Pat. No. 12,097,225, both of which are hereby incorporated by reference herein.

### TECHNICAL FIELD

The present invention relates to the field of probiotic compositions. The present field of invention is also related to the treatment and prevention of microbial pathogens pertaining to the infection of the genital and reproductive system. The microbial pathogens included and intended to be treated are diverse aerobic and anaerobic bacterial pathogens of the human vagina. The present invention also includes within its scope the prevention and treatment of vaginal yeast infections through the probiotic compositions disclosed in this present invention.

### BACKGROUND

The human vagina is a unique warm and moist chamber that is home to a considerable number of microbial populations. A healthy adult female vaginal microflora includes several kinds of bacteria and yeast and other microbiota of lesser-known species. The vaginal microbiome contains predominantly the lactic acid producing Lactobacillus. Lactobacillus iners, Lactobacillus crispatus, Lactobacillus gasseri, Lactobacillus jenesenii, followed by Lactobacillus acidophilus, Lactobacillus bulgaricus, Lactobacillus rhamnosus, Lactobacillus leichmanni, Lactobacillus vaginalis, Lactobacillus plantarum, Lactobacillus casei are some of the major Lactobacillus bacteria found in a healthy human vagina of pre-menopause age. This list is non-exhaustive in its nature. Other bacterial species colonizing the human vagina might include other lactic acid producing bacterial species from the genera Leptorichia, Leuconostoc, Atopobium, Streptococcus instead of Lactobacillus but Lactobacillus remains to be the most common of all and the most preferred to retain a healthy state. It is well established that all ethnic populations of the world possess one or the other kind of lactic acid producing Lactobacillus predominant bacteria inside the vagina. Almost all of the vaginal microbiota found in the vaginal epithelium are gram positive in nature. Production of lactic acid inhibits various pathogenic invasions in the vagina. Lactic acid producing bacteria also produce hydrogen peroxide that inactivates deadly viruses such as Human Immunodeficiency Virus, Herpes Simplex virus, or other pathogenic bacteria like Trichomoas vaginalis, Gardnerella vaginalis, or other bacteria those responsible for bacterial vaginosis. There are of course other species of bacteria too, found in the vagina but their numbers are negligible. Lactic acid producing bacteria control the over-growth of these other bacterial population and maintains the optimum count of different bacteria in the vagina. Production of lactic acid by Lactobacillus is responsible for maintaining a low acidic pH 4.5, preferably lesser, thereby discouraging other bacteria from thriving there thus acting as a natural barrier to unwanted microbial imbalances inside the vagina. The optimum vaginal pH ensures prevention of unwanted microbial flora such as yeast and different other bacteria other than Lactobacillus. In normal circumstances, a healthy human vagina is considered self-sufficient to retain its healthy PH levels. The healthy Lactobacillus count is replenished automatically if supported by abundance of glycogen eventually broken down to complex sugars along with proper diet and a healthy lifestyle.

The natural microbial population of human vagina also includes a limited population of yeast cells in its native state. Candida albicans, typically a fungus is also a natural inhabitant of the vagina in negligible quantities. Although C. albicans is the only dominant species from the Candida genus that is found in the vagina, other common species include C. glabrata, C. parapsilosis, C. tropicalis, and C. krusei. It is opined that vaginal yeast shares a commensal relationship with its host. Yet, sometimes Candida spp. have also been termed as an endo-symbiont playing an indirect role in maintenance of a balanced vaginal microbial ecosystem. Being the natural inhabitants of the vaginal ecosystem, it is proposed to compete with other undesirable fungal invaders within the vagina at the first place. Therefore, a balanced population of vaginal bacterial and yeast cells is desirable however, it is also known that an increase in Candida albicans population is responsible for vaginal yeast infections (vulvovaginitis) as well.

It is inherently a challenge to retain a natural balance of the vaginal microbial ecosystem. There is always a competition prevailing between the bacterial and the yeast population within the female genital system. Such an imbalance is obvious due to several direct or indirect factors. Hormonal imbalances resulting from excess stress in today's lifestyle in almost all adult females is one major reason of an imbalanced microbial population in their vagina. Stress also contributes to the imbalance of secretion of various female reproductive hormones. As such, a female undergoes fluctuations in hormones during her varying phases of menstrual cycle. For example, increases in estrogen levels are common prior to occurrence of menstrual bleeding. Such hormonal imbalance contributes to disturbing the natural balance of vaginal ecosystem. In addition, use of contraceptive devices, sexual intercourse, lifestyle disorders like diabetes, and use of various antibiotics ultimately have been shown to adversely affect the natural balance of a healthy vaginal microbiota predominantly affecting and reducing the count of healthy Lactobacillus from the vagina. This leads to various symptomatic or asymptomatic changes in the general course of life such as excessive vaginal discharge with a fishy odor such as leucorrhoca, vaginal itching, etc, that disturbs the general flow of life causing irritation and other associated social difficulties. These also may lead to severe pathogenic infections such as bacterial vaginosis, aerobic vaginitis, as well as severe yeast or fungal infections, which are most of the time asymptomatic. Present technical research on this subject have also established that occurrence or presence of such pathogenic infections within the female reproductive system also increases the chances of contracting other severe sexually transmitted infections ranging from human immunodeficiency virus, hepatitis, human papillomavirus, Neisseria gonorrhoeae, and Chlamydia trachomatis among others. Therefore, pathogenic infections of the vagina or other female genital tract of the vagina invites a huge risk in terms of contracting AIDS or cancer or other deadly sexually transmitted diseases. Hence, pathogenic infections of the human female genitalia are the doorstep of life-threatening diseases. An early detection, prevention and treatment of such pathogens is always necessary before it paves the way to such deadly diseases. Generally, treatment of vaginal pathogens has been performed with antibiotics such as metronidazole and clindamycin (for bacterial vaginosis), fluconazole, amphotericin-B (as antifungal) or others but it is an acknowledged fact that antibiotic treatment further contributes to the imbalance of inherent vaginal bacteria since antibiotics completely kill all kinds of bacteria present in the vagina including the lactic acid producing bacteria which is necessary for maintaining a good and healthy vaginal microbial ecosystem. The extensive use of antibiotics kills the probiotic Lactobacillus population of the vagina and sets an opportunity of overgrowth of vaginal yeast particularly and most commonly Candida albicans. An overgrowth of vaginal yeast is very common and undetectable. Increase in the vaginal yeast count is naturally reversible if proper caution is exercised. However, vaginal yeast infections (termed as Vaginal Candidiasys) demand treatment with antifungal drugs, which otherwise if left untreated impose serious medical complications provided the infections also enter the blood stream.

Chronic yeast infections of the vagina are also very common wherein the infection comes back even after treatment with antifungal drugs (fluconazole). Recurring yeast infections are caused due to several risk factors such as pregnancy, birth control pills or contraceptives, estrogen therapy, regular antibiotic use, diabetes and conditions that affect the immune system. Therefore, it becomes necessary to undergo treatment against recurring yeast infections repeatedly and multiple times which is devastating for a female of any given age. The problem of recurring yeast infection is more intense if caused by other Candida species, such as C. glabrata. Present antifungal treatment with mycostatin vaginal creams, vaginal gels containing the antifungals amphotericin B and flucytosine might not be totally effective at all in case of other yeast infections. None of these treatments have been reported to offer a permanent solution to the recurrence of vaginal yeast infections. Therefore, vaginal yeast infections are a major concern for all women across the world particularly those who fall under the age group of 15-45. Furthermore, in vitro results suggest the use of alternative agents other than antifungal fluconazole when treating vulvovaginitis caused by non-albicans species (especially C. glabrata or C. krusei) as these non albicans strains have been reportedly found to be not susceptible to fluconazole (Sandra S Richter et. al., Antifungal Susceptibilities of Candida Species Causing Vulvovaginitis and Epidemiology of Recurrent Cases, J Clin Microbiol. 2005 May; 43 (5): 2155-2162). Availability of such alternative agents are scarce and not commercially available.

Referring to bacterial infections of the vagina, Bacterial vaginosis (BV) is the most commonly known and the most frequent vaginal infection characterized by an imbalanced vaginal flora due to deficiency of lactobacilli. Bacterial vaginosis affects about 15-50% of reproductive aged women globally. Symptoms of BV are existence of a thin, homogeneous discharge and a strong vaginal odour, however most women with BV are unaware that they are the victims of BV. BV has also been associated with significant gynaecologic and obstetric complications including pelvic inflammatory disease, endometritis, and post-operative infections including post-partum endometritis. Besides, women also become prone to pre-term delivery, miscarriage, and amniotic fluid infections and an increased susceptibility to Human Immunodeficiency Virus (HIV) as well. Gardnerella vaginalis is known to be the most commonly invading pathogen to cause BV among other pathogens such as Atopobium vaginae, Bacteriodes spp, Molbiluncus, Megasphera, Mycoplasma hominnis, Peptostreptococcus, Prevotella being particularly prevalent as well. Other infections due to aerobic bacteria includes pathogenic strains of E. coli, Enterococcus faecalis, and Staphylococcus saprophyticus, Staphylococcus aereus, and Group B Streptococcus. Aerobic pathogenic infections of the vagina are termed as Aerobic Vaginitis (AV) which is distinct from BV. A study of 2015 reports that aerobic vaginitis requires treatment based on microscopy findings and a combined local treatment with any of the following which may yield the best results: antibiotic (infectious component), steroids (inflammatory component), and/or estrogen (atrophy component) (Donders G G et. al., Selecting anti-microbial treatment of aerobic vaginitis, Curr Infect Dis Reports. 2015May; 17 (5): 477). Unlike BV, AV does not respond well to metronidazole. Clindamycin is therefore considered to be a better choice against AV. Fluoroquinolones, such as ciprofloxacin and ofloxacin, have been used in treatment because they have little effect on the normal flora allowing for a rapid recovery from AV (Evelyn Kaambo et. al., Vaginal Microbiomes Associated With Aerobic Vaginitis and Bacterial Vaginosis, published online 2018 Mar. 26.doi: 10.3389/fpubh.2018.00078).

The presently known treatments of bacterial vaginosis in general, have been achieved with antibiotics such as metronidazole, clindamycin etc. or lactate gels. But it is reported that 20% to 30% of women with BV relapse within 1-3 months following standard antibiotic treatment. Besides it is also widely accepted that antibiotics completely wash off natural Lactobacillus bacteria as well, thereby washing off or depleting the natural microflora along with the pathogens. This leaves and provides enough scope for all pathogenic microbes to dominate the vaginal epithelial lining once again before the natural lactobacilli are able to re-colonize the vagina naturally. Irregularities and non-completion of the entire medication schedule by the subjects increases the possibilities of re-colonization by the pathogens earlier than the local Lactobacillus as well. Additionally, there also lie possibilities of recurrence of the pathogens due to descent from the gut, sexual intercourse, and other susceptible factors. Therefore, investigations and studies have been already conducted to check BV through the application of probiotics that may be effective in improved treatment or prevention of BV. L. rhamnosus GR-1 and L. fermentum B-54 have been studied as local vaginal instillations to reduce BV (G. Reid et. al., “Instillation of Lactobacillus and stimulation of indigenous organisms to prevent recurrence of urinary tract infections” Microecology and Therapy, vol. 23, pp. 32-45, 1995). Some of the other known probiotics to check BV are reported. Lactobacillus acidophilus which was found as effective treatment with trimethoprim/sulfamethoxazole. (See, S. J. Lee et. al. “Probiotics prophylaxis in children with persistent primary vesicoureteral reflux”, Pediatric Nephrology, vol. 22, no. 9, pp. 1315-1320, 2007). Another study involving oral probiotic delivery of combined compositions of L. rhamnosus GR-1 and L. reuteri RC-14 probiotic group delivered a lactobacilli-dominated normal vaginal microbiota restored from a BV vaginal flora to 37% of the patients as compared to 13% of the placebo patients. However, a different strain of L. rhamnosus GG was conclusively found to be ineffective in treating or preventing BV (G. Reid et. al., “Oral probiotics can resolve urogenital infections” FEMS Immunology & Medical Microbiology, vol. 30, no. 1, pp. 49-52, 2001). In another research involving combination of Lactobacillus gasseri 335 and Lactobacillus brevis in a vaginal tablet adversely affected the adhesion of G. vaginalis and the count of pathogenic G. vaginalis was also significantly reduced (S. Boris et. al., “Adherence of human vaginal lactobacilli to vaginal epithelial cells and interactions with uropathogens”, Infection and Immmunity, vol. 66, no. 5, pp. 1985-1989, 1994). A successful investigation was reported that would confront Candida albicans (yeast) and BV infections of the vagina through regular and periodic intake of probiotic yogurt containing Lactobacillus acidophilus, (E. Shalev et. al., “Ingestion of yogurt containing Lactobacillus acidophilus compared with pasteurized yogurt as prophylaxis for recurrent candidal vaginitis and bacterial vaginosis” Archives of Family Medicine, vol. 5, no. 10, pp. 593-596, 1996).

All the above studies have shown potential of using primarily lactic acid producing Lactobacillus either singly or in combination with different Lactobacillus strains to treat BV. But there is also evidence available wherein many other Lactobacillus strains have been found ineffective in treatment of BV. As per a review article on Vaginal Microbiota and the Use of Probiotics (Sarah Cribby et. al. Interdisciplinary Perspectives on Infectious Diseases, volume 2008, Article ID 256490), various other studies on treatment against bacterial vaginosis with various other probiotic Lactobacilli acidophilus strains were found to be completely ineffective as well. A probiotic formulation of L. fermentum, L. gasseri and L. rhamnosus was not successful in treating bacterial vaginosis. Furthermore, there also remains very rare chances of developing endocardiditis and bacterimia caused by lactobacilli. The review by Sarah Cribby et. al. prescribes prior proper documented evidence of using a specific Lactobacillus strain to be coined as suitable probiotic to combat BV since the mode of action of Lactobacillus bacteria was found to be highly strain specific. It could be concluded that not all Lactobacillus bacteria could function as a suitable probiotic against BV.

Although lactobacilli are naturally occurring bacteria present within a woman's vagina, overgrowth of lactobacilli might be responsible for causing cytolytic vaginosis (also known as Lactobacillus overgrowth syndrome) that disrupts the normal vaginal environment and becoming more acidic, excessive vaginal itching that irritates and causes inflammation of the vaginal walls, and the vulva, pain during urination and sexual intercourse and elevated vaginal discharge. Sometimes, excessive vaginal discharge symptoms are confused with either bacterial vaginosis or candidiasis. Cytolytic vaginosis is becoming more common in subjects with high glucose serum levels. Treatment of cytolytic vaginosis has been suggested with treatment of vaginal douche with sodium bicarbonate (Anupama Suresh et al., Indian J Sex Transm Dis AIDS. 2009 January-Jun; 30 (1): 48-50).

Therefore, it is proposed that there exists a high risk of development of cytolytic vaginosis due to treatment of bacterial vaginosis conditions with only Lactobacillus strains because of overgrowth of lactobacilli in the vagina. This will lead to further complications, as in such cases, it would be mistakenly concluded to increase BV rather than identification of cytolytic vaginosis. In another aspect, since it is factually established that the specific lactobacilli resident in each individual and different communities of the world are diverse, treatment with different external strains of lactobacilli which is not common or was not priorly existing in such subjects may be not functional. That is to say, a probability that a specific strain of lactobacilli acting as a probiotic in one particular group or individual against BV may not be applicable as a probiotic in a different individual or a different community. In other words, it is advisable to develop such probiotic formulations that would augment the naturally existing lactobacilli strain previously existing in the vagina of the specific subject (called doderlein vaginal microflora), rather than supplying different other strains of external Lactobacillus through other Lactobacillus based probiotic formulation(s). This would also have the advantage of avoiding the risk of developing cytolytic vaginosis also, since such probiotic formulation would intentionally not include any lactobacilli strain yet act as a biological catalyst to restore natural Lactobacillus microflora in the human vagina.

Additionally, it is the need of the hour to develop a single probiotic formulation that would treat and prevent aerobic vaginitis and as well as bacterial vaginosis along with simultaneous vaginal yeast infections as well with a broad spectrum application against any and all Candida species other than Canidida albicans. Such a new probiotic formulation would also avoid undergoing prior separate and specific diagnostic steps to identify the nature of vaginal infections thereby decreasing the cost of expense, and time of recovery beyond any doubt.

US2006/0217443A1 publishes use of gallic acid in a pharmaceutical formulation that is capable of inhibiting Trichomonas vaginalis, Gardnerella vaginalis, and Candida albicans but not inhibiting the growth of Lactobacillus acidophillus with potential to treat or prevent vaginal infections caused by these three selective organisms. However, there is no evidence provided in this patent application that gallic acid although not inhibiting the growth of Lactobacillus acidophilus is capable of promoting the growth of lactic acid bacteria. Furthermore, the effect of gallic acid alone on other species of Lactobacillus bacteria remains unknown. It is also not provided that gallic acid would inhibit the diverse microbial pathogens responsible for bacterial vaginosis as detailed above. Likewise, gallic acid alone would be unable to treat or prevent aerobic vaginitis pathogens and different Candida species.

WO2017/083549 further discloses a probiotic delivery system as a vaginal suppository composition comprising a pharmaceutically acceptable matrix that is solid at room temperature and in a dry environment but melts when comes in contact with the inner mucosa of the rectum or the vagina wherein the said matrix comprises a mixture of fatty acids selected from sodium bicarbonate, citric acid, Vitamin C, lauric acid, coconut oil, shea butter, and other pharmaceutically acceptable items such as polychylene glycol, hydrogels, cocoa butter, glycerinated gelatin, mineral oil, or shark liver oil. The composition of WO2017/083549 also includes one or more species of probiotic bacteria, and a pH stabilizing agent. The probiotic bacteria experimentally presented in the delivery system of this patent publication are L. acidophilus DDS 1, Bifidobacterium lactis, L. plantarum WCFSI, L. casei GG, L. rhamnosus GG, L. brevis, Bifidobacterium longum, L. salivarius, Streptococcus thermophilus, Bifidobacterium bifidum. Other theoretically disclosed probiotic bacteria that could be possibly included in this vaginal suppository are L. planetrium, L. casei, Bifidobacterium longum, L. lactis, Bacillus coagulans, L. bulgaricus, and L. gasseri. The suppository composition also includes a bunch of prebiotics and pH solubilizing agent(s) although no proper experimental details have been disclosed in this particular patent disclosure. There is no evidence of treatment against pathogenic infections of the vagina at all in this patent publication. Furthermore, there is lack of enablement of actual technical details, since combination of so many bacteria are also prone to experience inter-bacterial interference and competition. Therefore, such a formulation with so many bacterial types is not practically suggested.

Another patent publication US2018/0250318 discloses a composition particularly useful in maintaining and supporting healthy microflora in the female urogenital tract comprising a first therapeutic agent and a second therapeutic agent wherein the first therapeutic agent is a pentose or a disaccharide and the second therapeutic agent is an organic acid or selected from cyclodextrin, or a pectic substance or a non-digestible polysaccharide. Similar to the above prior art references, experimental details of actual treatment of vaginal infections including AV, BV and yeast infections are missing in this particular reference as well.

Thus, a review of the current state of the art lacks a proper probiotic measure to treat diverse pathogenic infections of the vagina. It would be highly appreciated to provide a single beneficial probiotic formulation that will be able to treat any kind of vaginal pathogens including a variety of pathogens causing bacterial vaginosis, or aerobic vaginitis, as well as different yeast infections of the vagina. Another definite advantage is such a formulation will prevent from further aggravating of the infections due to the delay caused in diagnosis. Such a formulation is desirable because this would avoid the delay of treatment and additional diagnostic measures to identify the specific nature of treatment on a particular subject thereby also decreasing the total cost of treatment as well. Hence, such a formulation would be easily affordable by the subjects who are more prone to vaginal infections due to sexual intercourse with multiple partners or genital exposure related to their profession. The present invention discloses a unique probiotic composition that successfully addresses the present drawbacks of all the existing methods of treatment against all known bacterial or yeast infections of the human female genital system ranging from vagina, or the anus, rectum and the like.

### SUMMARY

According to one embodiment of the invention, isolation of the infected vaginal swabs from different subjects followed by identification of diverse bacterial and fungal pathogens infecting the human vagina is provided.

According to another embodiment of the invention, various formulations at different concentrations that include the present composition of the present invention comprising Bacillus coagulans, Bacillus subtilis, citric acid and gallic acid are provided.

According to one more embodiment of the invention, the extent of protection measured in terms of zone of inhibitions obtained by various formulations at different concentrations that includes the present composition of the present invention comprising Bacillus coagulans, Bacillus subtilis, citric acid and gallic acid against the isolated vaginal pathogens are provided.

According to another embodiment of the invention, comparative growth of Bacillus coagulans and Bacillus subtilis in presence of the composition of the present invention have been provided.

According to yet another embodiment of the invention, comparative growth of inherent doderlein Lactobacillus species of the human vagina in presence of is also provided.

According to further embodiment of the invention, mechanism of action of the composition of the present invention is provided.

According to yet another embodiment of the invention, a composition comprising Bacillus coagulans, Bacillus subtilis, citric acid, gallic acid in the presence of excipients selected from bioadhesives, disintegrating agents, binders, humectants, anticaking agents, foaming agents, diluents, and carriers is also disclosed, wherein the bioadhesive is selected from carbomers, carbopol, polycarbophil, disintegrating agents are selected from beta-cyclodextrin, sodium bicarbonate, adipic acid, boric acid, sodium glycolate, binders are selected from starch and disaccharides, poly vinyl pyrollidone, humectants are selected from glycerine, aloevera, the anticaking agents are selected from silicon dioxide, sodium bicarbonate, and adipic acid. The list of excipients provided are in no-way limiting the scope of the present invention and is non-exhaustive in nature.

The invention is further described in terms of the non-limiting examples as provided herein below. A person skilled in the art would appreciate that any modifications or alterations of the invention as described herein below are to be construed well within its scope and ambit of the invention.

An embodiment of the invention discloses a composition for competitive inhibition of pathogens against but not limited to bacterial vaginosis, aerobic vaginitis, vaginal yeast and fungal pathogenic infections without the need of prior diagnosis thereof and restoration of microbial ecological balance comprising: (a) Bacillus coagulans; (b) Bacillus subtilis; (c) citric acid; and (d) gallic acid, including any combinations thereof.

An embodiment of the invention discloses a composition comprising Bacillus coagulans and Bacillus subtilis including any combinations thereof.

An embodiment of the invention discloses a composition comprising Bacillus coagulans, citric acid, and gallic acid including any combinations thereof.

An embodiment of the invention discloses a composition comprising Bacillus subtilis, citric acid, and gallic acid including any combinations thereof.

An embodiment of the invention discloses a composition wherein Bacillus coagulans is present at a concentration of up to 1 billion colony forming units (cfu), and Bacillus subtilis is present at a concentration of up to 1 billion colony forming units (cfu), citric acid between 10 mg to 100 mg and gallic acid between 10 mg to 100 mg per unit dose.

An embodiment of the invention discloses a composition wherein Bacillus coagulans is present at a concentration of 250 million cfu to 550 million cfu, preferably between 350 million cfu to 550 million cfu; Bacillus subtilis is present at a concentration of 250 million cfu to 550 million cfu, preferably between 350 million cfu to 550 million cfu; citric acid between 10 to 50 mg and gallic acid between 10 to 50 mg per unit dose.

An embodiment of the invention discloses a composition further including Bacillus laterosporus up to 1 billion colony forming units (cfu) per unit dose.

An embodiment of the invention discloses a composition further comprising excipients selected from bioadhesives, disintegrating agents, binders, humectants, anticaking agents, foaming agents, diluents, and carriers.

An embodiment of the invention discloses a composition wherein the bioadhesive is selected from carbomers, carbopol, polycarbophil, disintegrating agents are selected from beta-cyclodextrin, sodium bicarbonate, adipic acid, boric acid, sodium glycolate, binders are selected from starch and disaccharides, poly vinyl pyrollidone, humectants are selected from glycerine, and aloevera, and the anticaking agents are selected from silicon dioxide, sodium bicarbonate, and adipic acid.

An embodiment of the invention discloses a composition provided in any of the forms selected from a gel, ointment, cream, tablet, capsule, pessary, foam, washes, tampons, applicators, pads, for use as oral, or topical, or any site-specific mode of application.

An embodiment of the invention discloses a composition wherein Bacillus coagulans and Bacillus subtilis are present in the form of spores which germinates into vegetative forms as soon as contacted with the internal vaginal environment or the dermis optionally aided by the presence of citric acid and gallic acid.

An embodiment of the invention discloses a composition is stable for at least 2 years at temperatures up to 40° C. in absence of cold chain storage conditions.

An embodiment of the invention discloses a composition, and a method of competitive inhibition of pathogens and restoration of inherent doderlein bacteria without administering any Lactobacillus bacteria from external sources thereby maintaining microbial ecological balance of the reproductive and genital organs particularly the vagina for treatment of bacterial vaginosis, aerobic vaginitis, vaginal yeast and fungal pathogenic infections without the need of prior diagnosis thereof using a composition comprising (a) Bacillus coagulans; (b) Bacillus subtilis; (c) citric acid; and (d) gallic acid, including any combinations thereof, further wherein Bacillus coagulans and Bacillus subtilis is are present at a concentration up to 1 billion cfu in the form of spores, citric acid between 10 mg to 100mg and gallic acid between 10 mg to 100 mg per unit dose.

An embodiment of the invention discloses a method of prevention and reduction of recurrence of pathogenic infections using a composition comprising (a) Bacillus coagulans; (b) Bacillus subtilis; (c) citric acid; and (d) gallic acid, including any combinations thereof, further wherein the growth of doderlein Lactobacillus bacteria in the presence of Bacillus coagulans and Bacillus subtilis is more than the growth of doderlein Lactobacillus bacteria without the presence of Bacillus coagulans and Bacillus subtilis.

### OBJECTIVES OF THE PRESENT INVENTION

One objective of the present invention is to replenish the natural vaginal microflora through the use of new probiotic compositions of the present invention.

Another objective of the present invention is to promote the growth of local lactobacilli inside the vagina of a respective subject through the use of new probiotic compositions that does not include an external source of Lactobacillus.

A further objective of the present invention is to provide a new probiotic composition for the treatment of bacterial vaginosis due to any and almost all known types of bacterial or fungal pathogens or any other infectious microorganism.

Another objective of the present invention is to provide a new probiotic composition that is capable to prevent and treat aerobic bacterial infections of the vagina causing aerobic vaginitis as well.

One more objective of the present invention is to overcome the present drawbacks, side effects or limitations of treatment of vaginal yeast infections including those caused by either Candida albicans as well as all other known types of pathogenic yeast or fungal species of the vagina.

One further objective of the present invention also includes the prevention and treatment of any and all bacterial infections such as bacterial vaginosis, aerobic vaginitis and yeast or fungal infections without the need of specific prior diagnosis of the nature of the vaginal infection.

One further objective of the present invention also includes the prevention and treatment of recurrent vaginal bacterial infections such as bacterial vaginosis, aerobic vaginitis and yeast or fungal infections.

The basic objective of the invention is to retain consistent healthy vaginal pH of between 3.5-4.5 that promotes healthy microbiota of the human vagina or any other genital or reproductive parts of the human female such as rectum or the anus or the inner endometrial microflora through administration of the probiotic compositions of the present invention.

It is also an objective of the present invention to enable and maintain a healthy balance of the human female reproductive system irrespective of intrinsic and extrinsic disturbances caused due to hormonal imbalances, mood swings, different other medications or antibiotic treatment, vaginal douches, menstrual cycles, sexual intercourse, multiple sexual partners etc. (non-exhaustive) with the probiotic compositions of the present invention.

### BRIEF DESCRIPTION OF THE FIGURES

FIG. 1 Panel A is a microscope view of the morphology of isolate VP03 on a growth SCDA plate; FIG. 1 Panel B is a reference morphology of Gardnerella vaginalis bacterial vaginal pathogen; FIG. 1 Panel C is a view of the colony morphology of isolate VP06 on a growth PDA plate, and FIG. 1 Panel D is a reference colony morphology of Candida albicans fungal vaginal pathogen.

### DETAILED DESCRIPTION

As enumerated above, unlike the other probiotic compositions provided in the prior art including Lactobacillus bacteria, the present invention does not include any Lactobacillus bacteria at all. The present probiotic composition includes particularly Bacillus coagulans and Bacillus subtilis. Presence of Bacillus coagulans produce and release lactic acid, which in turn promotes the growth of other Lactobacillus bacteria to thrive and multiply their counts exponentially. Therefore it acts as an in-situ producer of lactic acid thereby preventing growth of pathogenic E. coli and Streptococcus strains within the vagina, Furthermore, Bacillus cogaulans and Bacillus subtilits are bacteria which are already recognized to be probiotics of the digestive system, and imposes no serious health risks to be administered in the human reproductive system as well. Bacillus coagulans and Bacillus subtilis also act by producing effective bacteriocins that kill a wide ambit of pathogenic bacteria causing bacterial vaginosis. At the same time, the probiotic composition as a whole also bears the potential to combat other aerobic vaginitis-causing microbes and vaginal yeasts including Candida albicans including other fungal pathogens other than C. albicans as well. The probiotic composition of the present invention therefore is a major technical development in the domain of treatment of all kinds of female genital infections and hence avoids the requirement of specific prior diagnosis. The probiotic compositions of the present invention are therefore advantageous and act in a synergistic manner to address multiple requirements in a single formulation which are currently absent in the present state of the art. The probiotic compositions of the present invention are capable to be formulated as a vaginal suppository to be directly instilled internally within the vagina with or without the help of a vaginal applicator. Alternatively, the probiotic compositions of the present invention are capable to be formulated as a composition for oral administration which traverses down the digestive tract to finally settle in the internal genital environment. The probiotic compositions of the present invention can also be administered as a vaginal douche or any genital cleansing formulations or as a tampon, sanitary pads and napkins or the like.

The invention is further described in the non-limiting working examples as described below.

### EXAMPLE 1

### Isolation of Infectious Strains From the Human Vagina

I. Collection for Vaginal Swab Specimens: HiCulture™ Transport Swabs were used for collection of specimens. Carefully swab was inserted into vagina about 2 inches (5 cm) past the introitus and gently rotated the swab for 10 to 30 seconds. It was made sure that the swab touches the vagina walls so that moisture is absorbed by the swab and then swab was withdrawn without touching the skin. The capped swab with the sample was inserted till the bottom of the sample collection tube containing medium and the tube was capped firmly. All such specimens collected from a few subjects in the form of swabs obtained from the vagina of the said subjects were labeled and transported to the laboratory in a temperature controlled box. After the transportation, the specimens were inoculated in appropriate medium as soon as possible.

II. Enrichment of Specimens: The collected swabs were inoculated into Soybean Casein Digest Medium (SCDM) and Potato Dextrose medium (PDB). The mediums were kept under incubation at 37° C. for 24-48 hrs. Enrichment in SCDM and PDB enumerate the presence of bacteria and fungi/yeast respectively.

III. Isolation of vaginal pathogens: After incubation, 1 ml of enriched broth of SCDM and PDB were inoculated into sterile petri plate and then poured SCDA (Soyabean Casein Digest Agar) and PDA (Potato Dextrose Agar) in respective plates. After solidification of agar, plates were incubated at 37° C. for 24-72 hrs. Uninoculated control plates were kept for incubation along with inoculated plates to ensure sterility of media and plate preparation.

Once the incubation was over, plates were observed for different colony morphology and growth pattern. All the different colonies were identified from SCDA and PDA plate. Every isolated colony was re-streak on earlier prepared and pre-incubated sterile SCDA and PDA plates. Each colony was streak on single plate. All plates of SCDA are kept for incubation at 37° C. and PDA at 30° C. for 24-72 hrs.

### EXAMPLE 2

### Identification of Isolated Pathogens

All the colony morphology observed was recorded as per Bergey's manual of bacteriology and staining was performed for isolates. Three different fungal pathogens (identified as VP01, VP02, VP06) and three different bacterial pathogens (identified as VP03, VP04 and VP05) which were most commonly found to occur from the infectious swabs were individually cultured for further examination as detailed below.

### EXAMPLE 3

### Formulation of Anti-Vaginitis Probiotic Composition

Antimicrobial activities against isolated vaginal pathogens (i.e. VP01, VP02, VP06, VP03, VP04 and VP05) were individually screened and analyzed to conclude and find effective probiotic compositions.

### Example 3.1

Different combination of selected probiotics strains with variation in potency were formed and underwent potential antimicrobial effect against isolated vaginal bacterial and fungal pathogens. The extents of zone of inhibition in antimicrobial activity were considered for selection of formulation. Table 1 below describes the working of the invention. The probiotic compositions of the present invention comprise (a) Bacillus coagulans (B.C.); (b) Bacillus subtilis (B.S.); (c) citric acid (CA); (d) gallic acid (GA); (e) beta-cyclodextrin (adhesive); (f) allentoin and (g) a pH regulator (h) lanolin (i) polyethylene glycol (j) a cooling agent etc., and (k) a bioadhesive.

Except as otherwise provided, the embodiments of the present invention and the working examples disclosed herein were conducted with Bacillus coagulans strain designated as SNZ1969 and Bacillus subtilis strain designated as SNZ1972. Bacillus coagulans SNZ1969 is deposited with Microbial Type Culture Collection with number assigned as MTCC-5724. Bacillus subtilis SNZ1972 is deposited with Microbial Type Culture Collection with number assigned as MTCC-5980. Although the experiments have been conducted with Bacillus coagulans SNZ1969 and Bacillus subtilis SNZ1972, the efficacy of the compositions of the present invention is not confined to these specific strains of Bacillus coagulans and Bacillus subtilis. The present invention is also capable to inhibit pathogenic infections and restore inherent doderlein bacteria comprising any given strain of Bacillus coagulans and Bacillus subtilis.

TABLE 1

| | | ZONE OF INHIBITION (in mm) | | | | | |
|---|---|---|---|---|---|---|---|
| SL | | Fungal Pathogens | | | Bacterial Pathogens | | |
| No. | Formulation | VP01 | VP02 | VP06 | VP03 | VP04 | VP05 |
| 1 | Gallic Acid (GA) | 35 | 35 | 9 | n-i | 9 | 11 |
| 2 | Citric Acid (CA) | 41 | 30 | n-i | 10 | 11 | 19 |
| 3 | GA + CA | 35 | 38 | 15 | 13 | 15 | 22 |
| 4 | BS* + GA + CA | 44 | 39 | 22 | 18 | 19 | 21 |
| 5 | Clotrimazole | n-i | 9 | 17 | n-i | 8 | n-i |
| | (commercial | | | | | | |
| | formulation) | | | | | | |
| 6 | A* | 45 | 39 | 19 | 13 | 11 | 16 |
| 7 | B* | 43 | 38 | 17 | 16 | 19 | 19 |
| 8 | C* | 42 | 34 | 21 | 18 | 21 | 21 |
| 9 | D* | n-i | n-i | 20 | 9 | 8 | 11 |
| 10 | Bacillus subtilis | — | — | 40 | — | — | — |
| | (1 billion spores) | | | | | | |
| 11 | Bacillus coagulans | — | — | 26 | — | — | — |
| | (1 billion spores) | | | | | | |
| 12 | Bacillus laterosporus | — | — | 22 | — | — | — |
| | (1 billion spores) | | | | | | |

Table 1 shows Initial Zone of Inhibition studies with various probiotic formulations.

A* is a formulation that includes Bacillus subtilis (350 million spores), Bacillus coagulans (350 millison spores), Gallic acid (GA) (50 mg) and Citric Acid (CA) (25mg) and Bacillus laterosporus (350 million spores). B* is a formulation that included Bacillus subtilis (350 million spores), Bacillus coagulans (350 million spores), GA (50 mg) and CA (25 mg). C* is a formulation that include Bacillus subtilis (350 million spores), Bacillus coagulans (700 million spores), GA (50 mg) and CA (25 mg). Similarly D* is a formulation that included Bacillus coagulans (100 million spores) along with GA (50 mg), CA (25 mg) along with Boric Acid. VP01, VP02 and VP06 are suspected fungal pathogens determined on the basis of morphological characteristics. VP03, VP04 and VP05 are suspected bacterial pathogens determined on the basis of morphological characteristics. The term ‘n-i’ denotes that there was no zone of inhibition obtained against the particular pathogen at all in presence of our probiotic formulations or the individual components thereof. The places left blank in the above table mean that no experiment on those parameters were conducted at all.

### Example 3.2

Based on the initial experiments above, various new formulations at different concentrations of each of the components were prepared and the corresponding zone of inhibition studies were performed. The zones of inhibition were also examined for each individual components at the same concentration levels as present in the respective combined formulations.

The various ranges of concentrations of each component of the invention is disclosed in the Table 2 below.

TABLE 2

| | B. coagulans | B. subtilis | Citric Acid | Gallic Acid |
|---|---|---|---|---|
| Serial No. | (million) | (million) | (mg) | (mg) |
| 1. | 700 | 25 | 10 | 100 |
| 2. | 550 | 125 | 20 | 60 |
| 3. | 350 | 350 | 20 | 40 |
| 4. | 125 | 550 | 60 | 20 |
| 5. | 25 | 700 | 100 | 10 |

Table 2 shows Various Concentration Ranges of the Individual Components.

The various formulations made with the above-mentioned different concentration ranges of the components mentioned in Table 2, are presented in Table 3 below.

TABLE 3

| Formulations | A | B | C | D | E |
|---|---|---|---|---|---|
| B. cogulans | 700 | 550 | 350 | 125 | 25 |
| (in million) per unit dose | | | | | |
| B. subtilis | 25 | 125 | 350 | 550 | 700 |
| (in million) per unit dose | | | | | |
| Citric Acid (in mg) per unit dose | 10 | 20 | 20 | 60 | 100 |
| Gallic Acid (in mg) per unit dose | 100 | 60 | 40 | 20 | 10 |

Table 3 shows Various Formulations prepared with each of the components at respective combination of concentration ranges.

The Zone of Inhibition were checked with the formulation A of Table 3, along with corresponding individual concentrations against each of the isolated pathogens VP01, VP02, VP06, VP03, VP04 and VP05 presented in Table 4 below.

TABLE 4

| Formulation | | | | | | |
|---|---|---|---|---|---|---|
| (B.c & B.s in million cfu, | ZONE OF INHIBITION (in mm) | | | | | |
| CA & GA in mg) | VP01 | VP02 | VP06 | VP03 | VP04 | VP05 |
| A | 11 | 13 | 19 | 15 | 15 | 15 |
| [B.c (700) + B.s (25) + | | | | | | |
| CA (10) + GA (100)] | | | | | | |
| B. coagulans (700) | n-i | 25 | n-i | 7 | 9 | 10 |
| B. subtilis (25) | 12 | 13 | 16 | n-i | 9 | 11 |
| CA (10) | n-i | n-i | n-i | n-i | n-i | n-i |
| GA (100) | 19 | 16 | n-i | 8 | 10 | 7 |

Table 4 shows Zone of Inhibition for Formulation A against individual components at the same concentrations thereof.

From the above Table 4, it is to be noted that in case of VP06, identified as Candida albicans, a fungal vaginal pathogen causing vaginal yeast infections, none of the components except Bacillus subtilis could inhibit VP06 but the combined formulation A shows greater extent (19 mm) of protection against VP06, as against only Bacillus subtilis (16 mm). Similarly, as against VP03, identified as Gardnerella vaginalis, a bacterial vaginal pathogen causing bacterial vaginosis, Bacillus subtilis and Citric Acid is unable to cause any inhibitory effect and Bacillus coagulans and Gallic acid shows negligible zone of inhibitions (7 & 8 respectively). Yet, the formulation shows substantial increase in the magnitude of zone of inhibition (15). Increase in the zone of inhibition is found to be subsistent as against VP06, VP03, VP04 and VP05 while acted by the combined formulation as against in presence of individual components.

The Zone of Inhibition were checked with the formulation B of Table 3, along with corresponding individual concentrations against each of the isolated pathogens VP01, VP02, VP06, VP03, VP04 and VP05 presented in Table 5 below.

TABLE 5

| Formulation | | | | | | |
|---|---|---|---|---|---|---|
| (B.c & B.s in million cfu, | ZONE OF INHIBITION (in mm) | | | | | |
| CA & GA in mg) | VP01 | VP02 | VP06 | VP03 | VP04 | VP05 |
| B | 14 | 16 | 25 | 16 | 16 | 14 |
| [B.c (550) + B.s (125) + | | | | | | |
| CA (20) + GA (60)] | | | | | | |
| B. coagulans (550) | n-i | 28 | n-i | 7 | 8 | 9 |
| B. subtilis (125) | 13 | 14 | 24 | n-i | 11 | 11 |
| CA (20) | n-i | n-i | n-i | 7 | n-i | n-i |
| GA (60) | 17 | 15 | n-i | 5 | 10 | 7 |

Table 5 shows Zone of Inhibition for Formulation B against individual components at the same concentrations thereof.

From the above Table 5, it is to be noted that in case of VP06, none of the components except Bacillus subtilis could inhibit VP06 but the combined formulation B shows increase (25 mm) of protection against VP06, as against only Bacillus subtilis (24mm). The formulation shows substantial increase in the magnitude of zone of inhibition in case of VP03, VP04 and VP05 as against the zone of inhibition caused by the individual components. Increase in the zone of inhibition is found to be subsistent as against VP06, VP03, VP04 and VP05 while acted by the combined formulation as against in presence of individual components.

The Zone of Inhibition were checked with the formulation C of Table 3, along with corresponding individual concentrations against each of the isolated pathogens VP01, VP02, VP06, VP03, VP04 and VP05 presented in Table 6 below.

TABLE 6

| Formulation | | | | | | |
|---|---|---|---|---|---|---|
| (B.c & B.s in million cfu, | ZONE OF INHIBITION (in mm) | | | | | |
| CA & GA in mg) | VP01 | VP02 | VP06 | VP03 | VP04 | VP05 |
| C | 16 | 17 | 23 | 17 | 17 | 15 |
| [B.c (350) + B.s (350) + | | | | | | |
| CA (20) + GA (40)] | | | | | | |
| B. coagulans (350) | n-i | 17 | n-i | 6 | 8 | n-i |
| B. subtilis (350) | 13 | 13 | 26 | 8 | 13 | 12 |
| CA (20) | 23 | n-i | n-i | 8 | 10 | 8 |
| GA (40) | 16 | 15 | n-i | 7 | 9 | 6 |

Table 6 shows Zone of Inhibition for Formulation C against individual components at the same concentrations thereof.

From the above Table 6, the formulation shows substantial increase in the magnitude of zone of inhibition in case of VP03 (17), VP04 (17) and VP05 (15) as against the negligible zone of inhibition caused by the individual components. Increase in the zone of inhibition is found to be subsistent as against VP02, VP03, VP04 and VP05 while acted by the combined formulation as against in presence of individual components.

The Zone of Inhibition were checked with the formulation D of Table 3, along with corresponding individual concentrations against each of the isolated pathogens VP01, VP02, VP06, VP03, VP04 and VP05 presented in Table 7 below.

TABLE 7

| Formulation | | | | | | |
|---|---|---|---|---|---|---|
| (B.c & B.s in million cfu, | ZONE OF INHIBITION (in mm) | | | | | |
| CA & GA in mg) | VP01 | VP02 | VP06 | VP03 | VP04 | VP05 |
| D | 21 | 20 | 25 | 17 | 14 | 12 |
| [B.c (125) + B.s (550) + | | | | | | |
| CA (60) + GA (20)] | | | | | | |
| B. coagulans (125) | n-i | 13 | n-i | 5 | 7 | n-i |
| B. subtilis (550) | 12 | 12 | 26 | 9 | 12 | 12 |
| CA (60) | 22 | n-i | n-i | n-i | 10 | 8 |
| GA (20) | 16 | 18 | N-i | n-i | 9 | 6 |

Table 7 shows Zone of Inhibition for Formulation D against individual components at the same concentrations thereof.

Especially in the case of above formulation D shown above, the magnitude of protection obtained individually by Bacillus coagulans (5) and Bacillus subtilis (9) against VP03 are minimal. The individual components Citric acid and Gallic Acid is not active against VP03 at all. However, there is a substantial increase in the protection obtained in case of the combined formulation D (17 mm). Thus, there is substantial increase obtained by the combined formulation as against each of the components when present individually. Increase in the zone of inhibition is found to be subsistent as against VP02, VP03 and VP04 while acted by the combined formulation as against in presence of individual components.

The Zone of Inhibition were checked with the formulation E of Table 3, along with corresponding individual concentrations against each of the isolated pathogens VP01, VP02, VP06, VP03, VP04 and VP05 presented in Table 8 below.

TABLE 8

| Formulation | | | | | | |
|---|---|---|---|---|---|---|
| (B.c & B.s in million cfu, | ZONE OF INHIBITION (in mm) | | | | | |
| CA & GA in mg) | VP01 | VP02 | VP06 | VP03 | VP04 | VP05 |
| E | 22 | 22 | 27 | 15 | 15 | 13 |
| [B.c (25) + B.s (700) + | | | | | | |
| CA (100) + GA (10)] | | | | | | |
| B. coagulans (25) | n-i | 13 | n-i | 6 | 7 | 6 |
| B. subtilis (700) | 13 | 13 | 27 | 9 | 13 | 13 |
| CA (100) | 25 | n-i | n-i | 9 | 11 | 10 |
| GA (10) | 13 | n-i | n-i | n-i | 7 | 6 |

Table 8 shows Zone of Inhibition for Formulation E against individual components at the same concentrations thereof.

In the case of above formulation E shown above, the magnitude of protection obtained individually by Bacillus coagulans (13) and Bacillus subtilis (13) against VP02 are minimal. The individual components Citric acid and Gallic Acid is not active against VP02 at all. However, there is a substantial increase in the protection obtained in case of the combined formulation E (22 mm). Thus, there is substantial increase obtained by the combined formulation as against each of the components when present individually. Increase in the zone of inhibition is found to be subsistent as against VP02, VP03 and VP04 while acted by the combined formulation as against in presence of individual components.

The Zone of Inhibition were checked with all the formulations A to C of Table 3, against each of the isolated pathogens VP01, VP02, VP06, VP03, VP04 and VP05 is presented in Table 9 below.

TABLE 9

| Formulations | | | | | | |
|---|---|---|---|---|---|---|
| (B.c & B.s in million cfu, | ZONE OF INHIBITION (in mm) | | | | | |
| CA & GA in mg) | VP01 | VP02 | VP06 | VP03 | VP04 | VP05 |
| A | 11 | 13 | 19 | 15 | 15 | 15 |
| [B.c (700) + B.s (25) + | | | | | | |
| CA (10) + GA (100)] | | | | | | |
| B | 14 | 16 | 25 | 16 | 16 | 14 |
| [B.c (550) + B.s (125) + | | | | | | |
| CA (20) + GA (60)] | | | | | | |
| C | 16 | 17 | 23 | 17 | 17 | 15 |
| [B.c (350) + B.s (350) + | | | | | | |
| CA (20) + GA (40)] | | | | | | |
| D | 21 | 20 | 25 | 17 | 14 | 12 |
| [B.c (125) + B.s (550) + | | | | | | |
| CA (60) + GA (20)] | | | | | | |
| E | 22 | 22 | 27 | 15 | 15 | 13 |
| [B.c (25) + B.s (700) + | | | | | | |
| CA (100) + GA (10)] | | | | | | |

Table 9 shows Zone of Inhibition of all the Formulations A to E against VP01 to VP06 pathogens.

The present formulation with Bacillus subtilis, Bacillus coagulans, Gallic Acid and Ciric Acid kills all the different infectious pathogens VP01 to VP06. However, the presently available commercial formulations could merely kill only selective pathogens. Bacillus subtilis has also shown wide spectrum activity especially against different fungal pathogens. Although different combinations containing Bacillus subtilis, gallic acid, and citric acid does show fair amount of broad-spectrum activity against most of the pathogens, presence of only these three components (i.e. Bacillus subtilis, gallic acid, and lactic acid) lacks continuous supply of lactic acid inside the vagina once the initial content of these three components are exhausted. Bacillus coagulans produces lactic acid and therefore continues to increase or replenish the vaginal microbial population with the native doderlein Lactobacillus species of the specific subject. This is phenomenal in also preventing the recurrence of the vaginal infectious pathogens in the long run. This is also explained in the following examples.

### EXAMPLE 4

Growth Profile of Bacillus coagulans and Bacillus subtilis in vaginal formulation at various pH greater than pH 7.

The pH of an infected vagina tends to be alkaline somewhere between pH 7-10. These experiments show that, within a span of 6 hours after exposure of the composition in contact of internal vaginal environment the pH comes down to pH 5-6 thereby turning the pH to acidic, and suitable to grow Bacillus coagulans and Bacillus subtilis, followed by Doderlein Bacteria.

Each of Bacillus subtilis (BS) and Bacillus coagulans (BC) present initially each at 350 million spores and the vaginitis formulation (VF) of the present invention comprising Bacillus subtilis (350 million spores) and Bacillus coagulans (350 million spores), along with citric acid and gallic acid were supplied with the following medium for measuring the growth of the individual bacteria present individually or in formulation (VF).

TABLE 10

| | | Yeast | | |
|---|---|---|---|---|
| Glucose | Peptone | Extract | | |
| (%) Carbon | (%) Nitrogen | (%) Growth | KH₂PO₄ | MgSO₄ |
| source | source | factor | (mg/100 mL) | (mg/100 mL) |
| 2 | 0.5 | 0.5 | 5 | 1 |

Table 10 provides Medium for growth of Bacillus subtilis and Bacillus coagulans.

TABLE 11

| pH profile | pH at 6^{th} Hr |
|---|---|
| BS | 5.77 (initial pH 6) |
| BC | 6.14 (initial pH 6) |
| VF | 5.20 (initial pH 6) |
| BS | 5.31 (initial pH 8) |
| BC | 7.05 (initial pH 8) |
| VF | 5.28 (initial pH 8) |
| BS | 6.00 (initial pH 10) |
| BC | 6.82 (initial pH 10) |
| VF | 5.66 (initial pH 10) |

Table 11 shows Change in pH after 6 hours of the medium from initial pH >6 (pH 6, 8 and 10 respectively) post inoculation of BS and BC in comparison with the Vaginal Formulation (VF).

From the above table 11, it is shown that both BC and BS individually can bring down the pH to acidic, by producing lactic acid while they grow in-vitro or in-vivo. However, the drop in pH is always more (in all the cases from pH 6, 8 and 10) for the vaginal formulation (VF) as compared to the BS and BC.

Furthermore, comparative growth profile of the individual bacteria (BS and BC) in comparison with corresponding vaginal formulation at specific pH 7 and pH 8 up to 24 hours is also provided below (Table 12 and 13).

The following Tables 12 and 13 show Growth profiles (OD at 600 nm) at pH 7.0 and pH 8.0, respectively.

TABLE 12
At pH 7.0

| | | | | | BS + BC | |
|---|---|---|---|---|---|---|
| | | Only BS | | Only BC | Without | BS + BC |
| Time | | with | | with | GA and | with |
| (Hr) | BS | GA, CA | BC | GA, CA | CA | GA, CA |
| 3 | 0.028 | 0.074 | 0.022 | 0.032 | 0.073 | 0.089 |
| 6 | 0.107 | 0.119 | 0.073 | 0.103 | 0.136 | 0.165 |
| 9 | 0.134 | 0.150 | 0.099 | 0.143 | 0.167 | 0.210 |
| 12 | 0.150 | 0.248 | 0.108 | 0.176 | 0.174 | 0.278 |
| 15 | 0.182 | 0.260 | 0.168 | 0.198 | 0.245 | 0.384 |
| 18 | 0.226 | 0.336 | 0.338 | 0.476 | 0.447 | 0.521 |
| 21 | 0.295 | 0.375 | 0.458 | 0.587 | 0.643 | 0.701 |
| 24 | 0.398 | 0.479 | 0.621 | 0.663 | 0.792 | 0.915 |

Table 12 above shows Growth profile at pH 7 (CA is Citric Acid & GA is Gallic Acid).

From the above Table 12, it is evident that the growth is higher in case of combined formulation (BS+BC+CA+GA) as against only Bacillus subtilis, or only Bacillus coagulans or each present with citric acid and gallic acid at the end of 24 hours starting from initial pH of 7.

TABLE 13
At pH 8.0

| | | | | | BS + BC | BS + BC |
|---|---|---|---|---|---|---|
| Time | | BS with | | BC with | Without | with |
| (Hr) | BS | GA, CA | BC | GA, CA | GA, CA | GA, CA |
| 3 | 0.073 | 0.086 | 0.0543 | 0.054 | 0.055 | 0.058 |
| 6 | 0.092 | 0.118 | 0.072 | 0.097 | 0.083 | 0.119 |
| 9 | 0.114 | 0.122 | 0.102 | 0.155 | 0.166 | 0.176 |
| 12 | 0.195 | 0.162 | 0.122 | 0.188 | 0.168 | 0.199 |
| 15 | 0.260 | 0.205 | 0.139 | 0.207 | 0.225 | 0.249 |
| 18 | 0.281 | 0.268 | 0.243 | 0.384 | 0.289 | 0.359 |
| 21 | 0.318 | 0.357 | 0.323 | 0.404 | 0.485 | 0.562 |
| 24 | 0.414 | 0.564 | 0.399 | 0.441 | 0.594 | 0.760 |

Table 13 shows Growth profile at pH 8 (CA is Citric Acid & GA is Gallic Acid).

From the above Table 13, it is evident that the growth is higher in case of combined formulation (BS+BC+CA+GA) as against only Bacillus subtilis, or only Bacillus coagulans or each present with citric acid and gallic acid at the end of 24 hours starting from initial pH of 8.

### EXAMPLE 5

Growth of inherent vaginal bacteria (Doderlein Bacteria) in presence of Bacillus coagulans.

To show the enhanced growth of doderlein vaginal bacteria (for example Lactobacillus gasseri and Lactobacillus acidophilus) in presence of BS+BC (as they are present in the present vaginal formulation) it is shown that the growth of doderlein bacteria (i.e. Lactobacillus gasseri and Lactobacillus acidophilus) is higher as compared when they are grown individually without the presence of Bacillus coagulans and Bacillus subtilis, thereby completely eliminating the possibilities of recurrence of infections by the pathogens due to increased growth of the inherent doderlein bacteria.

TABLE 14

| | Growth Profile (CFU/100 ml) after 24 hours | |
|---|---|---|
| Organism | Without BS + BC | With BS + BC |
| Lactobacillus gaseri | 30 × 10⁸ | 39 × 10⁸ |
| (initial concentration | pH 4.36 | pH 4.34 |
| 07 × 10⁸ CFU/100 ml) | | |
| Lactobacillus acidophilus | 15 × 10⁸ | 21 × 10⁸ |
| (initial concentration | pH 4.66 | pH 4.74 |
| 07 × 10⁸ CFU/100 ml) | | |

Table 14 shows Comparative growth of Lactobacillus gasseri and Lactobacillus acidophilus in presence of combination of Bacillus subtilis and Bacillus coagulans.

The above table 14 concludes that, (i) at least 30% increase in growth of Lactobacillus gasseri is seen when present in proximate with vaginal formulation than only presence of Lactobacillus gasser . (ii) at least 40% increase in growth of Lactobacillus acidophilus is seen when present in proximate with vaginal formulation than only presence of Lactobacillus acidophilus.

### EXAMPLE 6

### Formulations and Stability

Following formulations have been prepared to test the stability in terms of potency of the spores of Bacillus coagulans and Bacillus subtilis for a period of at least 6months at 40° C.

TABLE 15
Formulation 1 along with excipients

| Formulation 1 | Potency | |
|---|---|---|
| B. coagulans | 1 | Billion |
| B. subtilis | 1 | Billion |
| Citric acid | 15 | mg |
| Gallic acid | 40 | mg |

| | |
|---|---|
| Carbopol | q.s |
| Carboxymethyl cellulose | q.s |
| Polycarboxylic acid | q.s |
| Lactose | q.s |
| Boric acid | q.s |

Formulation 1 along with excipients

TABLE 16

| Formulation 1 | Potency | Initial | 3 Months | 6 Months |
|---|---|---|---|---|
| B. coagulans | 1 × 10⁹ | 1029 msg* | 1017 msg | 1008 msg |
| B. subtilis | 1 × 10⁹ | 1045 msg | 1028 msg | 1013 msg |

*msg: million spores per gram

Table 16 shows Stability of Formulation 1 in terms of potency of Bacillus coagulans and Bacillus subtilis at 40° C.

TABLE 17
Formulation 2 along with excipients

| Formulation 2 | Potency | |
|---|---|---|
| B. coagulans | 1 | Billion |
| B. subtilis | 1 | Billion |
| Citric acid | 20 | mg |
| Gallic acid | 20 | mg |

| | |
|---|---|
| Polycarbophill | q.s |
| Beta Cyclodextrin | q.s |
| Sodium bicarbonate | q.s |
| Adipic acid | q.s |
| Boric Acid | q.s. |

Formulation 2 along with excipients

TABLE 18

| Formulation 2 | Potency | Initial | 3 Months | 6 Months |
|---|---|---|---|---|
| B. coagulans | 1 × 10⁹ | 1023 msg* | 1008 msg | 1012 msg |
| B. subtilis | 1 × 10⁹ | 1056 msg | 1027 msg | 1007 msg |

*msg: million spores per gram

Table 18 shows Stability of Formulation 2 in terms of potency of Bacillus coagulans and Bacillus subtilis at 40° C.

The results show that there is little or minimum loss of potency of the Bacillus coagulans and Bacillus subtilis for a span of 6 months at 40° C. Therefore, it is concluded that the formulations comprising Bacillus coagulans, Bacillus subtilis, citric acid, gallic acid in combination with other excipients are stable and therefore are stable for a period of at least 2 years without the need of any cold chain storage requirements.

It may also be noted that the excipients as shown are non-exhaustive in nature. The invention includes in its scope additional formulations that may be potentially developed by adding other excipients as per the need and mode of application of the formulation. Mere replacement of excipients or addition of additional components in addition to Bacillus coagulans, Bacillus subtilis, citric acid, gallic acid, including a combination thereof is to be construed to be within the legitimate ambit and scope of the present invention.

### EXAMPLE 7

Mechanism of Action of the Probiotic Formulation of the present invention.

A healthy vagina is normally acidic with pH 3-4. When Bacterial vaginosis (BV) occurs, it is caused due to a mixed pathogenic and fungal infections. These pathogens and fungi colonize the vagina and do so by turning the environment from acidic to alkaline (pH >7). This shift to alkalinity strongly favors the pathogenic and fungal species and seriously impairs colonization by healthy bacteria (particularly beneficial lactobacilli species prevalent in the human vagina). The composition as presented in the form of the above-mentioned formulations in Example 3 are all designed to disrupt and kill pathogenic fungal and bacterial strains which is already established, further shift of pH to acidic encourage recolonization by healthy vaginal flora. After the formulation is brought in contact with the vaginal environment, the acidifiers particularly the citric acid and gallic acid begin their action by reducing the pH from greater than 7 (in case of infected vagina). Further, citric acid and gallic acid along with the available moisture content inside vaginal environment induces conversion of the spores of Bacillus coagulans and Bacillus subtilis to germinate as well, and the said probiotic bacteria start growing inside the vagina. Once the pH begins reaching less than 5-6, the bacterial strains begin germination. As a part of this germination, growing Bacillus coagulans' and Bacillus subtilis secrete bacteriocins, lactic acid, short chain fatty acids, anti-fungal compounds, and nutrient factors. These bacterial strains (including metabolites produced by the strains on site) in combination with the Gallic acid provide broad spectrum action against the infection-causing organisms. Thus, the site is now cleared for the return of normal vaginal flora. Bacillus coagulans and Bacillus subtilis both will not permanently colonize the vagina but will act as transient colonizers. However, during this transient period, these probiotic's metabolites will also promote growth of Lactobacillus acidophilusand Lactobacillus gasseri (native to a healthy vagina also called Doderlein bacteria). This leads to restoration of normal healthy vaginal flora and also the risk of recurrence of pathogenic infections in the vagina is severely reduced because the present composition promotes growth of doderlein bacteria or the native vaginal bacteria.

As compared with traditional antibiotic treatment, the step of restoring the healthy vaginal flora is completely ignored since antibiotics kill all good and bad bacteria thereby making room for the pathogens to invade again before the inherent native doderlein bacteria starts colonizing the vagina.

Additionally, the colonization of Bacillus coagulans and Bacillus subtilis is only temporary. Both Bacillus coagulans and Bacillus subtilis produce lactic acid. Production of lactic acid encourages replenishment and growth of native Lactobacillus species within the vagina which is inherent bacteria of the vagina. Therefore, the present composition automatically supplements the specific inherent bacteria which was originally present in the vagina of the specific subject. Thus, the present composition helps in the re-colonization of specific doderlein bacteria and not some other external probiotic as available according to the present state of the art. Due to the continuous availability of lactic acid, Bacillus coagulans and the Bacillus subtilis slowly are outnumbered by doderlein lactic acid bacteria of the Lactobacillus species.

Synergistic effects of the components of the compositions of the present invention as demonstrated by inhibition zone studies and methods used detailed in Examples 8-9 below.

### EXAMPLE 8

IDENTIFICATION OF ISOLATED PATHOGENS VP03 AND VP06. As shown above in Tables 4-9 of Example 3.2, the data demonstrate that compositions of the present invention inhibit pathogens VP03, identified as Gardnerella vaginalis a bacterial vaginal pathogen causing bacterial vaginosis, and pathogen VP06, identified as Candida albicans, a fungal vaginal pathogen causing vaginal yeast infections and which may also be involved in bacterial vaginosis. These data show that compositions of the present invention inhibit vaginal pathogens Gardneralla vaginalis and Candida albicans causing bacterial vaginosis and vaginal yeast infections, respectively, the latter also considered a fungal pathogenic infection. The inhibition of these pathogens by the compositions of the invention are demonstrated by the synergistic effect of the components of the present inventionBacillus coagulans, Bacillus subtilis, citric acid, and gallic acid as shown by the inhibition zone studies as detailed in the following Examples below.

VP03: Growth Media used: SCDA for isolating bacterial cultures from infected vaginal swabs. Vaginal swabs obtained from an infected patient were grown on Soyabean Casein Digest media, and thereafter single colonies were re-streaked on Soyabean Casein Digest Agar plates to obtain isolated colonies of a single bacteria, which were designated as VP03, VP04 and VP05. Soyabean Casein Digest medium is a common medium usually practiced to isolate and culture various bacteria only.

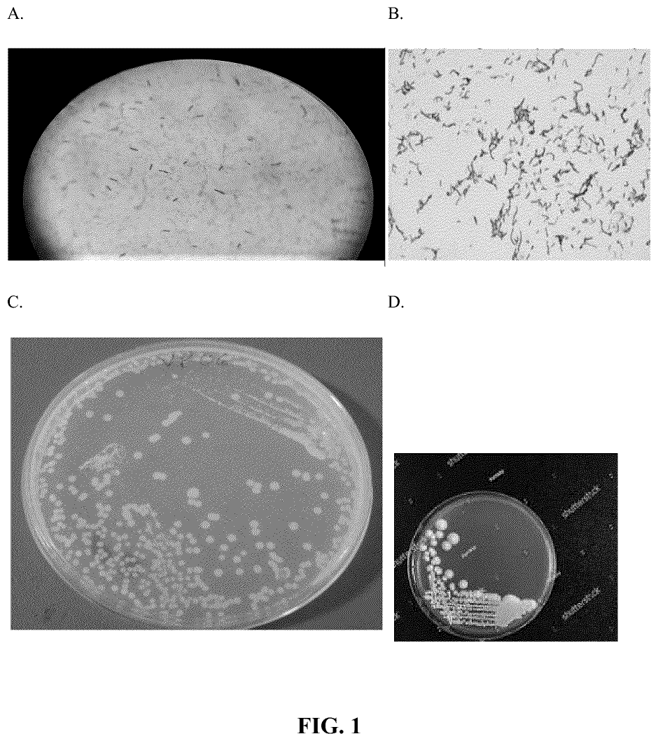

Observed morphology under microscope as shown in FIG. 1 panel A, of the culture of the designated pathogen VP03 grown and streaked on a Soybean Casein Digest Agar (SCDA) plate, matching with the morphology of a reference Gardnerella vaginalis shown in FIG. 1 panel B available in the public domain at https://microbe-canvas.com/Bacteria/grampositive-rods/cells-branched/facultative-anaerobic/catalase-negative-5/gardnerella-vaginalis.html. Therefore, the VP03 isolate is specifically most likely pathogenic bacteria Gardnerella vaginalis.

Vaginal swabs obtained from infected patients were grown on Potato Dextrose Agar (PDA) media, and thereafter single colonies were re-streaked on Potato Dextrose Agar plates to obtain fungal isolates, which were designated as VP06, VP02 and VP01. Potato Dextrose Agar is a common medium usually practiced to isolate and culture various fungi only, specifically applicable for yeast or fungi. As shown in FIG. 1 panel C the appearance of the VP06 isolate colony morphologies clearly resembles Candida albicans, the causative pathogen for vaginal yeast infections, as identifiable by its clear circular, round to oval shaped distinct colonies as shown in FIG. 1 panel D the reference morphology obtained from the public domain at https://www.shutterstock.com/search/yeast-potato-dextrose.

### EXAMPLE 9

### Study: Synergistic Growth of Doderlein Native Lactobacillus Bacteria in Presence of a Novel Probiotic Formulation Comprising Bacillus coagulans, Bacillus subtilis, Citric Acid & Gallic Acid Against Pathogens Infecting the Human Vagina

One objective of the present invention is to replenish the natural vaginal microflora with new probiotic compositions of the present invention. Another objective of the present invention is to promote the growth of local lactobacilli inside the vagina of a respective subject with new probiotic compositions that do not include an external source of Lactobacillus.

The composition: According to the various embodiments of the invention, various formulations comprising at different concentrations Bacillus coagulans, Bacillus subtilis, citric acid, and gallic acid are provided with their respective ranges as detailed in Table 19 below:

TABLE 19

| Formulations | A | B | C | D | E |
|---|---|---|---|---|---|
| B. cogulans | 700 | 550 | 350 | 125 | 25 |
| (in million cfu) per unit dose | | | | | |
| B. subtilis | 25 | 125 | 350 | 550 | 700 |
| (in million cfu) per unit dose | | | | | |
| Citric Acid | 10 | 20 | 20 | 60 | 100 |
| (in mg) per unit dose | | | | | |
| Gallic Acid | 100 | 60 | 40 | 20 | 10 |
| (in mg) per unit dose | | | | | |

Experimental Methods: 1) all the above 5 formulations were inoculated in 200 ml of MRS media taken in two sets each of 5 flasks (total 10) wherein the said MRS media was first adjusted to pH 9 by adding required amount (approximately 8 ml) of 2.0 M sodium hydroxide, to imitate alkaline vaginal pH when infected by any of the frequent bacterial or viral pathogens; 2) additional flasks containing the MRS media (1 for each set) were also taken and initially kept blank without any inoculation; 3) all the flasks were incubated for 24 hrs and Optical Density was measured and Plating was done on sterile MRS agar plates for cell counts CFU/ml to quantify the growth of Bacillus coagulans & Bacillus subtilis in the total 10 flasks in both the sets. Thereafter, each of the 5 flasks of both the sets (total 10) were autoclaved to heat kill the grown Bacillus coagulans & Bacillus subtilis in the total 10 flasks in both the sets.

Thereafter, in one set, all the 5 initial flasks (in which Bacillus coagulans & Bacillus subtilis was first grown and also including the one additional flask in which nothing was grown in the first step, were inoculated with viable culture (24 hours) of Lactobacillus acidophilus (10% v/v). Samples were withdrawn at 24 hours, from all the 6 flasks to measure the growth in terms of CFU/ml which would quantify the amount of Lactobacillus acidophilus grown without prior growth of Bacillus coagulans and Bacillus subtilis comprising the formulations A to E, as compared to the Lactobacillus acidophilus grown with prior growth of Bacillus coagulans and Bacillus subtilis comprising the formulations A to E. It was found that the amount of Lactobacillus acidophilus grown with the prior growth of Bacillus coagulans and Bacillus subtilis comprising the formulations A to E, was considerably and substantially higher than the growth of Lactobacillus acidophilus grown without or in absence of prior growth of Bacillus coagulans and Bacillus subtilis comprising the formulations A to E.

Thereafter, in another set, all the 5 initial flasks (in which Bacillus coagulans & Bacillus subtilis was first grown and also including the one other additional flask in which nothing was grown in the first step, were inoculated with viable culture (24 hours) of Lactobacillus gasseri (10% v/v). Samples were withdrawn at 24 hours from all the 6 flasks to measure the growth in terms of CFU/ml which would quantify the amount of Lactobacillus gasseri grown without prior growth of Bacillus coagulans and Bacillus subtilis comprising the formulations A to E, as compared to the Lactobacillus gasseri grown with prior growth of Bacillus coagulans and Bacillus subtilis comprising the formulations A to E. It was found that the amount of Lactobacillus gasseri grown with the prior growth of Bacillus coagulans and Bacillus subtilis comprising the formulations A to E, was considerably and substantially higher than the growth of Lactobacillus gasseri grown without or in absence of prior growth of Bacillus coagulans and Bacillus subtilis comprising the formulations A to E, as shown in Table 20 below.

TABLE 20

| | Blank | | | | | |
|---|---|---|---|---|---|---|
| | Growth | Formulation | Formulation | Formulation | Formulation | Formulation |
| | CFU/ | A | B | C | D | E |
| Hrs | 100 ml | Growth of Bacillus coagulans & Bacillus subtilis in CFU/100 ml | | | | |
| 0 | — | 2.5 × 10⁷ | 3.4 × 10⁷ | 5.5 × 10⁷ | 4.1 × 10⁷ | 2.8 × 10⁷ |
| 24 | — | 2.8 × 10⁸ | 3.0 × 10⁸ | 3.5 × 10⁸ | 3.1 × 10⁸ | 3.0 × 10⁸ |

AUTOCLAVED/HEAT KILLED Bacillus coagulans &
Bacillus acidophilus at 0 and 24 hours

Inoculated with Lactobacillus acidophilus in all the flasks, and then measured
growth of Lactobacillus acidophilus at 0 and 24 hours

| 0 | 7 × 10⁸ | 7 × 10⁸ | 7 × 10⁸ | 7 × 10⁸ | 7 × 10⁸ | 7 × 10⁸ |
|---|---|---|---|---|---|---|
| 24 | 15 × 10⁸ | 21 × 10⁸ | 31 × 10⁸ | 42 × 10⁸ | 34 × 10⁸ | 24 × 10⁸ |
| | Blank | | | | | |
| | Growth | Formulation | Formulation | Formulation | Formulation | Formulation |
| | CFU/ | A | B | C | D | E |
| Hrs | 100 ml | Growth of Bacillus coagulans & Bacillus subtilis in CFU/100 ml | | | | |
| 0 | — | 5.5 × 10⁷ | 5.9 × 10⁷ | 6.5 × 10⁷ | 6.1 × 10⁷ | 5.6 × 10⁷ |
| 24 | — | 2.5 × 10⁸ | 3.3 × 10⁸ | 3.9 × 10⁸ | 3.0 × 10⁸ | 2.7 × 10⁸ |

AUTOCLAVED/HEAT KILLED Bacillus coagulans &
Bacillus subtilis

Inoculated with Lactobacillus gasseri in all the flasks, and then measured
growth of Lactobacillus gasseri at 0 and 24 hours

| | | | | | | |
|---|---|---|---|---|---|---|
| 0 | 7 × 10⁸ | 7 × 10⁸ | 7 × 10⁸ | 7 × 10⁸ | 7 × 10⁸ | 7 × 10⁸ |
| 24 | 30 × 10⁸ | 39 × 10⁸ | 40 × 10⁸ | 41 × 10⁸ | 38 × 10⁸ | 37 × 10⁸ |

The experimental procedures as mentioned in the paragraphs to were repeated with different other formulations wherein these compositions were prepared using a different (i.e. other than the proprietary strain of Bacillus coagulans SNZ 1969® of the applicant) strain Bacillus coagulans (MTCC25197) and a different (i.e. other than the proprietary strain of Bacillus subtilis SNZ 1972® of the applicant) strain Bacillus subtilis (MTCC25199). The formulations prepared are provided in the table 21 below:

TABLE 21
Formulations with other strains of Bacillus coagulans &
Bacillus subtilis

| Formulations | A1 | B1 | C1 | D1 |
|---|---|---|---|---|
| B. cogulans | 350 | 125 | 550 | 350 |
| (in million cfu) per unit dose | | | | |
| B. subtilis | 350 | 550 | 125 | 350 |
| (in million cfu) per unit dose | | | | |
| Citric Acid | 20 | 20 | 30 | 40 |
| (in mg) per unit dose | | | | |
| Gallic Acid | 40 | 40 | 20 | 20 |
| (in mg) per unit dose | | | | |

The comparative results of the growth of the Lactobacillus strains in prior presence of the formulations Al to D1 as mentioned in Table 21 above, and in absence of formulations A1 to D1 are provided in the below mentioned tables 22 and 23:

TABLE 22
Comparative growth of Lactobacillus acidophilus in presence and
in absence of formulations Al to B1 comprising other strains of
Bacillus coagulans and Bacillus subtilis

| | | Formulation | Formulation | Formulation | Formulation |
|---|---|---|---|---|---|
| | | A1 | B1 | C1 | D1 |
| | | Growth in | Growth in | Growth in | Growth in |
| Hrs | Blank | CFU/ml | CFU/ml | CFU/ml | CFU/ml |
| 0 | — | 3.1 × 10⁷ | 5.0 × 10⁷ | 3.8 × 10⁷ | 2.2 × 10⁷ |
| 24 | — | 2.9 × 10⁹ | 4.0 × 10⁹ | 2.8 × 10⁹ | 2.5 × 10⁹ |

Growth of Lactobacillus acidophilus (in CFU/ml)

| | | | | | |
|---|---|---|---|---|---|
| 0 | 5.9 × 10⁸ | 5.9 × 10⁸ | 5.9 × 10⁸ | 5.9 × 10⁸ | 5.9 × 10⁸ |
| 24 | 12 × 10⁸ | 18 × 10⁸ | 25 × 10⁸ | 30 × 10⁸ | 22 × 10⁸ |

TABLE 23
Comparative growth of Lactobacillus gasseri in presence and in
absence of formulations Al to B1 comprising other strains of Bacillus
coagulanns and Bacillus subtilis

| | | Formulation | Formulation | Formulation | Formulation |
|---|---|---|---|---|---|
| | | A1 | B1 | C1 | D1 |
| | | Growth in | Growth in | Growth in | Growth in |
| Hrs | Blank | CFU/ml | CFU/ml | CFU/ml | CFU/ml |
| 0 | — | 4.3 × 10⁷ | 5.1 × 10⁷ | 4.8 × 10⁷ | 4.0 × 10⁷ |
| 24 | — | 2.7 × 10⁹ | 3.6 × 10⁹ | 3.3 × 10⁹ | 3.0 × 10⁹ |

Growth of Lactobacillus gasseri (in CFU/ml)

| | | | | | |
|---|---|---|---|---|---|
| 0 | 5.9 × 10⁸ | 5.9 × 10⁸ | 5.9 × 10⁸ | 5.9 × 10⁸ | 5.9 × 10⁸ |
| 24 | 20 × 10⁸ | 25 × 10⁸ | 35 × 10⁸ | 30 × 10⁸ | 26 × 10⁸ |

Thus, from the above results, it is established that the compositions of the present invention comprising any strains of Bacillus coagulans, Bacillus subtilis, citric acid and gallic acid are capable of ensuring synergistic growth of inherent doderlein vaginal Lactobacillus strains. The formulations with other strains of the Bacillus coagulans and Bacillus subtilis also produced higher growth of Lactobacillus acidophilus and Lactobacillus gasseri in prior presence of the formulations prepared with other strains of the Bacillus coagulans and Bacillus subtilis, as against in the absence of prior growth of Bacillus coagulans and Bacillus subtilis. Therefore, the present invention is not limited to the specific proprietary strains of Bacillus coagulans & Bacillus subtilis.

All the formulations A to E proved considerably increased and synergistic growth of different Lactobacillus probiotic bacteria native to the human vagina, as compared to the growth of the same Lactobacillus bacteria in absence of the said formulations A to E.

Inferences: All the formulations A to E ensure complete protection against these identified (although non-exhaustive) pathogen(s) infecting the human vagina. Further, in case of all the formulations, at least for on one occasion, the zone of inhibition by these formulations is greater than the sum of the zone of inhibitions by the individual constituents. Furthermore, in almost all occasions, all formulations A to E provide better and higher zone of inhibition(s) than the highest zone of inhibition provided by any of its respective constituent components. Additionally, the difference in zone of inhibitions by formulations A, B, & E against VP06, and formulation D against VP03 are statistically significant as well. The formulations A to E enable and facilitate re-growth and replenishment of doderlein Lactobacillus bacteria native specifically to any human vagina as compared to without the formulations A to E.

Based on the above inferences, it is observed that the above-mentioned formulations of A to E have markedly different characteristics with respect to capacity to inhibit pathogens and also with respect to cause rapid and increased growth of doderlein native vaginal bacteria. Additionally, it is observed that all the formulations have shown higher zone of inhibitions against the highest zone of inhibition shown by any one of the corresponding constituent components in almost all the experiments shown above. The foregoing data demonstrate synergism with respect to the zone of inhibitions of all the formulations A to E in at least one experiment for each of the formulations.

Therefore, without intending to be bound by any theory, the method of the present invention comprises the steps to: (1) Activate therapy via acidification of vagina due to the presence of citric acid and gallic acid; (2) Competitively inhibit and eliminate causative fungi and pathogens due to the bacteriocins released by BC and BS; and (3) Promote restoration of natural flora to prevent recurrence.

### Advantages of the Invention

Additionally, the present invention as disclosed in the above formulations comprising of Bacillus coagulans, Bacillus subtilis, citric acid and gallic acid could check all different types of bacterial and fungal pathogens infecting the vagina. Hence the necessity for specific diagnosis is no more essential to be performed thereby saving the cost and time of treatment.

Application of the probiotic compositions of the present invention does not need any separate administration of antibiotics for treatment of pathogenic infections of the vagina. Thus, it restricts development of antimicrobial resistance among the pathogens.

The combination of Bacillus subtilis and Bacillus coagulans in the present formulation is in the form of spores also containing citric acid and gallic acid compressed and converted into a tablet unlike all existing vaginal probiotic formulations which contain Lactobacillus strains in a vegetative form. Vegetative forms of the bacteria cannot sustain the high amount of pressure and load in the tablet formulation and therefore they are not stable, and their capacity to induce the desirable effect when administered in the vagina is highly compromised. These formulations also have a very short shelf life, and therefore require cold chain transportation. In contrast, the probiotic components of the formulations of the present invention are in the form of spores and hence they are more stable and have a better shelf life without the need of any cold chain transportation.

The present probiotic formulations intended for direct vaginal administration germinate within the vagina as soon as they get the required moisture content post administration. The citric acid and gallic acid content in the formulations act as synergistic initiators for effective germination of the spores. As explained earlier, these two bacilllus species i.e. B. subtilis and B. coagulans act as an in-situ producer of lactic acid, thereby allowing the doderlein Lactobacillus species to replenish and grow exponentially. Further, with passage of time, the count of B. subtilis and B. coagulans species reduces automatically with the growth of doderlein Lactobacillus bacteria as these two bacillus species are not the natural inhabitants of the vaginal microbial ecosystem. Therefore, this combined probiotic formulation encourages the competitive inhibition of pathogens and restoration of natural microbial ecological balance of the human vagina.

Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entireties for all purposes.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

We claim:

1. A composition for restoration of doderlein vaginal microbial ecological balance in a human subject, comprising Bacillus coagulans, Bacillus subtilis, citric acid and gallic acid,
wherein said Bacillus coagulans is present at a concentration between 25 million to 750 million colony forming units (cfu), Bacillus subtilis is present at a concentration between 25 million to 750 million cfu, said citric acid between 10 mg to 100 mg, and said gallic acid between 10 mg to 100 mg per unit dose,
wherein said citric acid and said gallic acid in the composition initially acidify the internal vaginal environment to promote germination of said Bacillus coagulans and said Bacillus subtilis in said composition thereby providing a continuous supply of lactic acid inside the vagina for restoration and growth of naturally occurring doderlein vaginal bacteria,
wherein said composition is provided in a dosage form selected from the group consisting of gel, ointment, cream, tablet, capsule, pessary, foam, washes, tampons, applicators, pads, and suppository.

2. The composition of claim 1, wherein the Bacillus coagulans is Bacillus coagulans SNZ 1969 and the Bacillus subtilis is Bacillus subtilis SNZ 1972.

3. A method of restoration of doderlein vaginal microbial ecological balance in a human subject a in need of treatment of a vaginal condition comprising the step of
administering a composition comprising Bacillus coagulans, Bacillus subtilis, citric acid, and gallic acid, wherein said Bacillus coagulans and Bacillus subtilis are present in the form of spores, and wherein said Bacillus coagulans is present at a concentration between 25 million to 750 million colony forming units (cfu), said Bacillus subtilis is present at a concentration between 25 million to 750 million cfu, citric acid between 10 mg to 100 mg and said gallic acid between 10 mg to 100 mg per unit dose.

4. The method of claim 3, wherein the Bacillus coagulans is SNZ 1969 and Bacillus subtilis is SNZ 1972.

5. The method of claim 3, wherein restoration of inherent vaginal doderlein bacteria in a human female is achieved without administering any Lactobacillus bacteria from external sources thereby restoring and maintaining microbial ecological balance of the vagina.

6. The method of claim 3, wherein the condition treated is selected from the group consisting of bacterial vaginosis, aerobic vaginitis, vaginal yeast, and fungal pathogenic infections.

7. The method of claim 3, wherein said vaginal condition is treated without requiring prior diagnosis thereof.

8. The method of claim 4 wherein said composition is provided in a dosage form selected from the group consisting of gel, ointment, cream, tablet, capsule, pessary, foam, washes, tampons, applicators, pads, and suppository.

9. A method of prevention and reduction of recurrence of human vaginal pathogenic infections comprising the step of
administering a composition comprising Bacillus coagulans, Bacillus subtilis, citric acid, and gallic acid, wherein the growth of doderlein Lactobacillus bacteria in presence of Bacillus coagulans and Bacillus subtilis is more than the growth of doderlein Lactobacillus bacteria without the presence of Bacillus coagulans and Bacillus subtilis,
wherein restoration of inherent vaginal doderlein bacteria in a human female is achieved without administering any Lactobacillus bacteria from external sources thereby restoring and maintaining microbial ecological balance of the vagina.

10. The method of claim 9 wherein the Bacillus coagulans is SNZ 1969 and Bacillus subtilis is SNZ 1972.

11. The method of claim 10 wherein said Bacillus coagulans and Bacillus subtilis are present in the form of spores, and wherein said Bacillus coagulans is present at a concentration between 25 million to 750 million colony forming units (cfu), said Bacillus subtilis is present at a concentration between 25 million to 750 million cfu, citric acid between 10 mg to 100 mg and said gallic acid between 10 mg to 100 mg per unit dose.

12. The method of claim 11, wherein the vaginal pathogenic infection treated is selected from the group consisting of bacterial vaginosis, aerobic vaginitis, vaginal yeast, and fungal pathogenic infections.

13. The method of claim 12 wherein said composition is provided in a dosage form selected from the group consisting of gel, ointment, cream, tablet, capsule, pessary, foam, washes, tampons, applicators, pads, and suppository.
